(19) United States
(12) Patent Application Publication
(10) Pub. No.: US 2003/0045604 A1
(43) Pub. Date: Mar. 6, 2003

(54) Dental root canal filling cones

(76) Inventor: Joachim E. Klee, Radolfzell (DE)

Correspondence Address: DENTSPLY INTERNATIONAL INC., 570 West College Avenue, York, PA, 17405-0872, US

(21) Appl. No.: 10/213,320

(22) Filed: Aug. 6, 2002

Related U.S. Application Data
(60) Provisional application No. 60/312,017, filed on Aug. 13, 2001.

(51) Int. Cl. A61K 6/08; C08L 1/00; C08K 3/00; C08J 3/00

(52) U.S. Cl. 523/116

(57) ABSTRACT

Dental root canal filling cones are prepared from a filler and a thermoplastic polymer, wherein the thermoplastic polymer is formed by polymerization of polymerizable diepoxide monomer and amine monomer. The amine monomers are primary monoamine and/or a disecondary diamine. The filler has about 40 to 90 weight-% of the cones providing a radio-opacity of at least 3 mm/mm aluminum.

### TECHNICAL FIELD

Described are dental root canal filling cones comprising: filler and thermoplastic polymer, wherein said thermoplastic polymer is formed by polymerization of polymerizable diepoxide monomer and amine monomer, said amine monomers being primary monoamine and/or a disecondary diamine, said filler comprising 40 to 90 weight-% of said cones providing a radio-opacity of at least 3 mm/mm aluminum.

### BACKGROUND OF THE INVENTION

In the last decades gutta-percha cones in combination with a root canal sealer are the most popular material used for root canal filling by master-point technique or by lateral condensation. The clinical success of a root canal filling depends on complete and tight filling. To improve the clinical success further and to make the root canal therapy more easily and safe both an excellent connection between root canal sealer and cavity wall on the one side and canal sealer and root canal cones must be achieved by new application technique.

Due to the polar hydrophilic moieties epoxide-amine basing root canal sealer adapt well to the cavity walls. Proofed is the tightness of a filled root canal by numerous studies. Moreover, recently, an adhesion of the epoxide-amine basing root canal sealer AH Plus (Dentsply De Trey) of 4 MPa was measured (J D Pécora et al., Braz. Dent. J. 12 (2001) 27) that shows how well the material is bonded to the cavity walls.

It is well-known that polymers of different polymer classes frequently are thermodynamically incompatible. That means they do not undergo any connection and they do not adhere well to each other. Same is the case for non-polar gutta-percha and the most of the more polar root canal filling materials. Consequently, a demand is to make root canal cones and root canal sealer more compatible.

In view of the further treatment, root canal sealer as well as root canal cones shell be thermoplastic to be removable if corrections are demanded or core build-up shell occur.

Recently, the first thermoplastic root canal sealer was invented (U.S. Pat. No. 5,624,976, 25.03.1994).

Recently, a thermosetting resin based material was applied for thermoset resin cones whereby either the root canal sealer or the cone are conductive ones (EP 0608361). There are some disadvantages for thermosetting cones, because they are:

difficult to remove

difficult to cut

not re-workable by thermal or solution processes

not suitable for injection molding, casting processes or related processes

not suitable for special filler treatment as described by this patent.

It is an object of the invention to provide a soluble and thermoplastic dental root canal cone that is easy to remove, that undergoes a connection to the thermoplastic sealer and which provides a radio-opacity of at least 3 mm/mm Al.

### DESCRIPTION OF THE PREFERRED EMBODIMENTS

Invented dental root canal filling cones comprise: filler and thermoplastic polymer, wherein said thermoplastic polymer is formed by polymerization of polymerizable diepoxide monomer and amine monomer, said amine monomers being primary monoamine and/or a disecondary diamine, said filler comprising 40 to 90 weight-% of said cones providing a radio-opacity of at least 3 mm/mm aluminum.

The dental root canal filling cones are composed of at least one thermoplastic polymer or they are composed of a thermoplastic polymer in the outer sphere of the cone and a core material in the inner sphere selected from the group of metals, ceramics, glass fibers or other thermoplastic or thermosetting plastic polymers such as polyamides, polyester, polyurethanes, polyethylene or polypropylene.

The thermoplastic polymers of the dental root canal filling cones are selected from the group of epoxide-amine addition polymers of the general formulas:

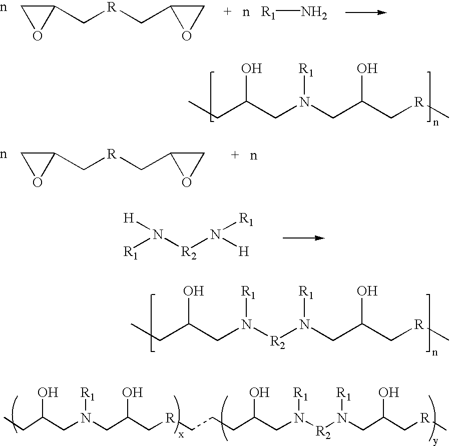

wherein R is a moiety formed from a diepoxide, such as

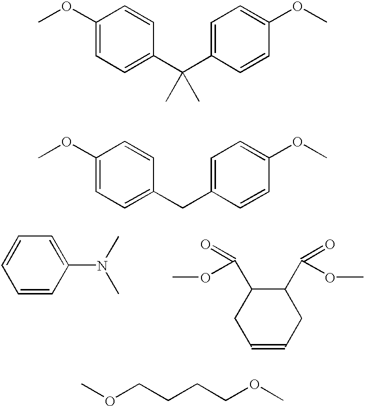

R₁ denotes a monofunctional substituted C₁ to C₁₈ alkylene, a substituted or unsubstituted C₅ to C₁₈ cycloalkylene, a substituted or unsubstituted C₅ to C₁₈ arylene or heteroarylene, such as

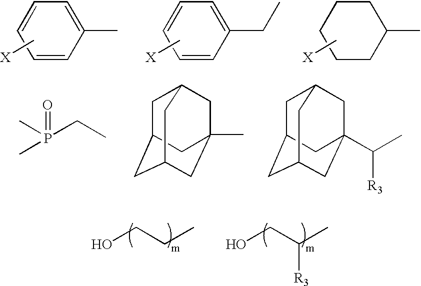

R₂ denotes a difunctional substituted or unsubstituted C₁ to C₁₈ alkylene, a substituted or unsubstituted C₅ to C₁₈ cycloalkylene, a substituted or unsubstituted C₅ to C₁₈ arylene or heteroarylene, such as

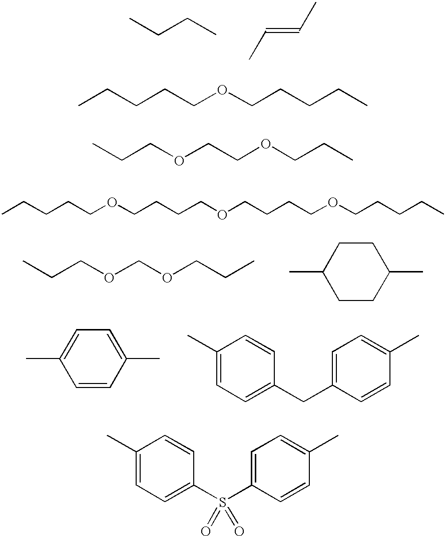

R₃ denotes hydrogen or C₁ to C₁₈ alkylene, such as H, CH₃, C₂H₅, C₃H₇ and X is hydrogen or a substituent selected from the group of OCH₃, F, Cl, Br, J, CH₃, COCH₃, NO₂, COOC₂H₅.

As epoxide monomer is used a diepoxide selected from the group of diglycidylethers such as diglycidyl ether of bisphenol-A, diglycidyl ether of bis-phenol-F, butandiol diglycidyl ether, N,N-diglycidylaniline or Δ³-tetrahydrophthalic acid diglycidyl ester.

Preferred amines are primary monoamines such as benzylamine, 1-aminoadamantan, α-phenethylamine and ethanol amine and disecondary diamines such as N,N′-dibenzyl ethylene diamine, N,N′-dibenzyl-3,6-dioxa-octandiamine-1,8, N,N′-dibenzyl-5-oxanonane diamine-1,9, N,N′-dibenzyl-(2,2,4)/(2,4,4)-trimethylhexamethylene diamine, N,N′-dicyclohexyl ethylene diamine, N,N′-dimethyl-p-xylylene diamine.

The achieve excellent mechanical properties and a high level of radio-opacity the dental root canal filling cones contains fillers such as inorganic compounds like La₂O₃, ZrO₂, BiPO₄, CaWO₄, BaWO₄, SrF₂, Bi₂O₃ or organic fillers, such as polymer granulate, splinter polymers or a combination of organic and/or inorganic fillers. Consequently, the Dental root canal filling cones provide a radio-opacity of at least 3 mm/mm Al, preferably at least 5 to 7 mm/mm Al, most preferably at least 7 mm/mm Al.

The process of preparation of dental root canal filling cones occurs by thermal addition polymerization of the diepoxide monomer and the amine monomer in presence of fillers and a simultaneous or a subsequent forming (casting) process of cones.

An other alternative is a two step-procedure, namely

i) thermal addition polymerization of the diepoxide monomer and the amine monomer on the filler surface

ii) forming (casting) process of the surface-modified filler of (i) by thermal and/or pressure processes.

### EXAMPLE 1

128.313 g (337.67 mmol) bisphenol-A diglycidyl ether (Mₙ 380 g/mol), 10.535 g (33.77 mmol) bisphenol-F diglycidyl ether, 28.140 g (185.72 mmol) 1-amino-adamantane, 63.241 g (185.72 mmol) N,N′-dibenzyl-5-oxanonanediamine-1.9 and 660.070 g CaWO₄, 165.018 g ZrO₂, and 9.980 g Aerosil 200 were mixed homogeneously and polymerized 24 hours at 60° C.

The composition is characterized by following values: radio-opacity RO=10.1 mm/mm Al glass transition temperature T_{g}=64° C. and volumetric shrinkage 1.13 vol.-%.

The obtained thermoplastic composite material was used for a thermal reforming process to form root canal cones.

### EXAMPLE 2

250.00 g (734.39 mmol) bisphenol-A diglycidyl ether, 22.255 g (146.88 mmol) 1-amino-adamantane, 200.059 g (587.51 mmol) N,N′-dibenzyl-5-oxanonanediamine-1.9 and 2249.112 g of a Barium-alumo silicate glass were mixed homogeneously and polymerized 24 hours at 60° C.

The composition is characterized by following values: radio-opacity RO=3.1 mm/mm Al, glass transition temperature T_{g}=37° C. and volumetric shrinkage 1.46 vol.-%.

I claim:

1. Dental root canal filling cones comprising: filler and thermoplastic polymer, wherein said thermoplastic polymer is formed by polymerization of polymerizable diepoxide monomer and amine monomer, said amine monomers being primary monoamine and/or a disecondary diamine, said filler comprising 40 to 90 weight-% of said cones providing a radio-opacity of at least 3 mm/mm aluminum.

2. Dental root canal filling cones of claim 1, composed of at least a thermoplastic polymer.

3. Dental root canal filling cones of claim 1, composed of a thermoplastic polymer in the outer sphere of the cone and a core material in the inner sphere selected from the group of metals, ceramics, glass fibers or other thermoplastic or thermosetting polymers such as polyamides, polyester, polyurethanes, polyethylene or polypropylene.

4. Dental root canal filling cones of claim 1, wherein said amine monomer and said epoxide monomer are polymerized to form polymer within the scope of at least one of the general formulas: 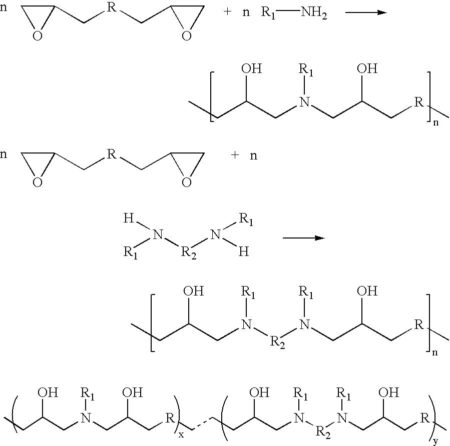
wherein R is a moiety formed from a diepoxide, selected from the group consisting of 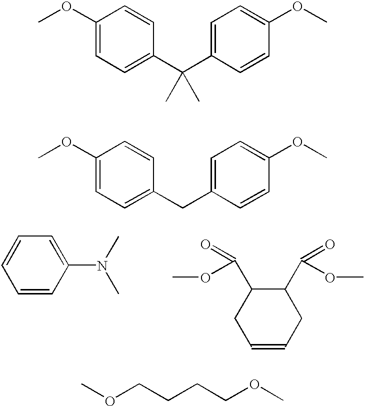
R₁ denotes a monofunctional substituted C₁ to C₁₈ alkylene, a substituted or unsubstituted C₅ to C₁₈ cycloalkylene, a substituted or unsubstituted C₅ to C₁₈ arylene or heteroarylene, selected from the group consisting of 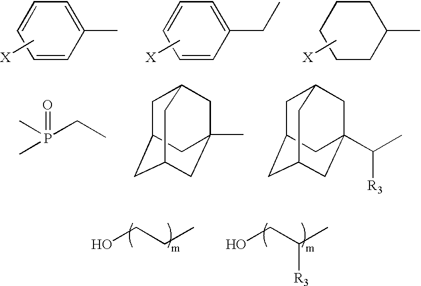
R₂ denotes a difunctional substituted or unsubstituted C₁ to C₁₈ alkylene, a substituted or unsubstituted C₅ to C₁₈ cycloalkylene, a substituted or unsubstituted C₅ to C₁₈ arylene or heteroarylene, selected from the group consisting of 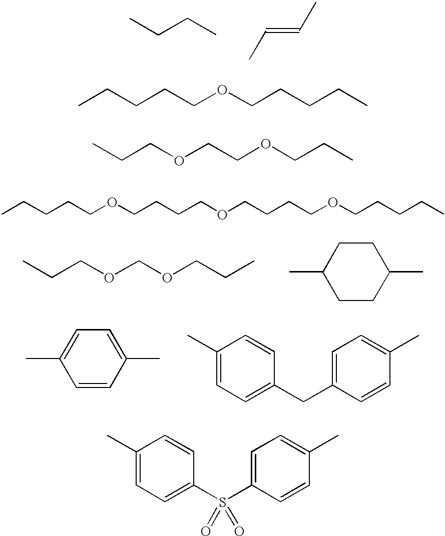
R₃ denotes hydrogen or C₁ to C₁₈ alkylene, such as H, CH₃, C₂H₅, C₃H₇ and X is hydrogen or a substituent selected from the group consisting of OCH₃, F, Cl, Br, J, CH₃, COCH₃, NO₂, COOC₂H₅.

5. Dental root canal filling cones of claim 1, wherein said epoxide monomer is a diepoxide selected from the group of diglycidylethers such as diglycidyl ether of bisphenol-A, diglycidyl ether of bis-phenol-F, butandiol diglycidyl ether, N,N-diglycidylaniline or Δ³-tetrahydrophthalic acid diglycidyl ester.

6. Dental root canal filling cones of claim 1, wherein said primary monoamine preferably is benzylamine, 1-aminoadamantan, α-phenethylamine and ethanol amine.

7. Dental root canal filling cones of claim 1, wherein said disecondary diamine preferably is N,N′-dibenzyl ethylene diamine, N,N′-dibenzyl-3,6-dioxa-octandiamine-1,8, N,N′-dibenzyl-5-oxanonane diamine-1,9, N,N′-dibenzyl-(2,2,4)/(2,4,4)-trimethylhexamethylene diamine, N,N′-dicyclohexyl ethylene diamine, N,N′-dimethyl-p-xylylene diamine.

8. Dental root canal filling cones of claim 1, wherein said filler is an inorganic compound such as La₂O₃, ZrO₂, BiPO₄, CaWO₄, BaWO₄, SrF₂, Bi₂O₃ or organic fillers, such as polymer granulate, splinter polymers or a combination of organic and/or inorganic fillers.

9. Dental root canal filling cones of claim 1, containing fillers which provide a radio-opacity of at least 3 mm/mm Al, preferably at least 5 to 7 mm/mm Al, most preferably at least 7 mm/mm Al.

10. Dental root canal filling cones of claim 1, wherein said cones are soluble in polar organic solvents such as CHCl₃, tetrahydrofurane or dimethyl formamide, said cones producible from those polymer solutions.

11. Dental root canal filling cones of claim 1, wherein said cones contain additives such as stabilizer and plasticizer.

12. Process for the preparation of dental root canal filling cones of claim 1, comprising: filler and thermoplastic polymer, wherein said thermoplastic polymer is formed by polymerization of polymerizable diepoxide monomer and amine monomer, said amine monomers being primary monoamine or a disecondary diamine, said filler comprising 40 to 90 weight-% of said cones providing a radio-opacity of at least 3 mm/mm aluminum characterized by a thermal addition polymerization of the diepoxide monomer and the amine monomer and a simultaneous or a subsequent thermally workable or processes.

13. Process for the preparation of dental root canal filling cones of claim 1, comprising the steps of
iii) thermal addition polymerization of the diepoxide monomer and the amine monomer on the filler surface; and
iv) forming (casting) process of the surface-modified filler of (i) by thermal and/or pressure processes.
